(19) United States
(12) Patent Application Publication
(10) Pub. No.: US 2012/0260681 A1
(43) Pub. Date: Oct. 18, 2012

(54) Cooling Plant in Particular for the Processing of Food and a method of Cleaning the Cooling Plant

(75) Inventors: Per Bruun Fammé, Odense C (DK); Hans Faltum, Otterup (DK)

(73) Assignees: Hans Faltum, Otterup (DK); CLEANSOLVE HOLDING APS, Odense C (DK); GRAM EQUIPMENT A/S, Vojens (DK)

(21) Appl. No.: 13/503,106

(22) PCT Filed: Oct. 19, 2010
(86) PCT No.: PCT/DK10/50276

§ 371 Date: Jul. 3, 2012

(30) Foreign Application Priority Data
Oct. 20, 2009 (DK) PA 2009 01140

(51) Int. Cl. F25B 49/00; A61L 2/18; A61L 2/24; F25B 47/00

(52) U.S. Cl. 62/78; 62/303

(57) ABSTRACT

The invention relates to a cooling plant an a method of cleaning such cooling plant. The cooling plant comprises a cooling chamber and at least one blower with a blast exit for circulating gas in the cooling chamber. The cooling plant further comprises an at least partly removable screen and a surface treatment liquid supply. The removable screen comprises a hole structure with a first and a second side and a plurality of holes extending from the first to the second side. The screen is arranged such that it totally or partly covers the blast exit in a first position so that at least a part of the gas supplied from the blast exit passes directly from the blast exit and through the hole structure from the first side thereof. The surface treatment liquid supply being arranged to supply a surface treatment liquid at least partly on the first side of said hole structure.

### TECHNICAL FIELD

The present invention relates to a cooling plant in particular for the processing of food. The cooling plant of the invention is simple and cost effective to clean. The invention also relates to a cooling plant system and a method of cleaning a cooling plant.

### BACKGROUND ART

Cooling plants are widely used in the food industry for cooling and freezing food. Industrial cooling plants are adapted to provide a fast cooling of the food. For hygienic reasons and for the quality of the food it is required that the food is cooled very rapidly. Food that remains at temperatures above 5° C. for too long is in high risk of being contaminated or subjected to growth of undesired bacteria and according to law and/or for health reasons it will often be required to destruct such food and furthermore for fast production the cooling time is essential. When freezing food the quality of the food is highly dependent of the freezing time and it is well known that a rapid freezing of the food provides the best quality. In other words in order to meet the requirements to hygiene and/or health and to obtain high productivity and high quality of food, the industrial cooling plants used today comprise an intensive blowing system to ensure fast cooling of the food.

The industrial cooling plants used today are normally not plants that are readily available, but most often they are tailer-made to meet specific requirements. Therefore the cooling plants are often different from each other both with respect to size and shape. Often the cooling plants are fairly large and comprise a cooling chamber with a plurality of elements, such as plates or conveyer bands for the food to be cooled.

For hygienic reasons the cooling chamber should be kept clean and free from bacteria and food remains.

Cooling plants are today normally cleaned manually where personnel enter the cooling chamber and clean the equipment manually or by the so called CIP (Clean-in-Place) method which includes one or both of the following principles:
Applying a cleaning solution as a low-energy spray to fully wet the surface and allowing the cleaning solution to work.
Applying a high energy impinging spray of water or cleaning solution in order to mechanically and optionally chemically clean the hard surfaces.

Elevated temperature and chemical detergents are often employed to enhance cleaning effectiveness.

The CIP method works fully acceptable seen from a hyginic point of view. However, the process is very expensive in that it either requires manually applying the liquid(s) or it requires expensive equipment for performing automatic or semi-automatic CIP, i.e. a large number of sprays need to be built into the cooling chamber of the cooling plant for performing an effective CIP process and usually large amounts of cleaning fluids are used. Furthermore, the CIP process is rather time consuming, in that the CIP process usually takes several hours, depending on the size of the cleaning chamber.

In order to provide an improved cleaning process DK patent 175817 suggests to use the blowing system of the cooling plant in the cleaning process by applying the cleaning solution on at least one inner surface of the cleaning chamber and allowing the blowing air generated by the blowing system to foam the cleaning solution and to distribute the foam to all surfaces in the cooling chamber. A similar method was described in an article in Levnedsmiddelbladet, 3, 2003, pages 14 and 18, by Per Bruun Famme. This article describes that an acceptable cleaning in particular of the cooling elements can be obtained in a spiral or flow freezer by applying the cleaning liquid on a conveyer band in the cooling chamber.

The method of cleaning a cooling chamber of a cooling plant using the blowing system of the cooling plant in the cleaning process as described above has for certain cooling chambers shown to be acceptably fast compared to the CIP method described above, and for cooling plants of a limited size and with a cooling chamber with a simple regular shape the method has shown to work acceptably. However, the method is not fully reliable and may leave non-cleaned spots or require a longer treatment time. Furthermore the method is not sufficiently effective for large cooling plant or cooling plants with cooling chambers of highly irregular shapes.

### DISCLOSURE OF INVENTION

The object of the invention is to provide an improved method of cleaning a cooling plant and a cooling plant adapted for performing such cleaning process in a simple and cost effective way.

This object has been solved by the invention as defined in the claims. The invention and embodiments of the invention provide additional benefits which will be clear from the claims and the description of the inventions as well as from the examples.

The term “cooling plant” is used herein to mean an industrial cooling plant for cooling down food and/or for freezing food. In other words, a cooling plant as used herein is adapted for removing heat from food and not merely keeping food cold. The cooling plant of the invention may preferably be a freezing plant adapted for freezing food, a chilling plant adapted for chilling food without freezing the food or a combination thereof.

The cooling plant of the invention comprises at least one cooling chamber and at least one blower comprising a blast exit for circulating gas in the cooling chamber. Generally it is desired that the cooling plant comprises a blower system comprising at least one blower and preferably several blowers as it is known in the art and as it will be clear from the description.

The cooling plant of the invention further comprises an at least partly removable screen and a surface treatment liquid supply. The term “removable” means that the screen can be removed partly or totally from the blast exit. The removable screen comprises a hole structure with a first and a second side and a plurality of holes extending from the first to the second side thereof. The screen is arranged such that in its first position it totally or partly covers the blast exit so that at least a part of the gas supplied from the blast exit when it is in operation passes directly from the blast exit and through the hole structure from the first side thereof. The term “passes directly” is used herein to mean that the gas supplied should pass in a substantially straight line from the blast exit and preferably not be a reflected gas consisting only of gas reflected from other surfaces of the cooling plant. The surface treatment liquid supply is arranged to supply a surface treatment liquid at least partly on the first side of the hole structure. The surface treatment liquid supply may in one embodiment be removable from the cooling plant. In another embodiment the surface treatment liquid supply is stationary in the cooling plant.

The gas may preferably be air, and in the following it will be described as air, but it should be understood that the air could be totally or partly replaced by other gas or gasses.

The cooling plant may be as any prior art cooling plant e.g. as described in U.S. Pat. No. 4,281,521, U.S. Pat. No. 5,452,588, U.S. Pat. No. 5,968,578, U.S. Pat. No. 6,583,181, US 2005/0138953, U.S. Pat. No. 7,178,356 and WO 01/56409 with the additional features comprising an at least partly removable screen and a surface treatment liquid supply as described herein.

The method of the invention has shown to be very effective in practice for cooling plants of any sizes and shapes, and furthermore it has surprisingly shown that the cleaning method of the invention for cleaning the cooling plant requires much less treatment liquid than the prior art method, which further adds to the cost reduction and additionally makes the method and the cooling plant of the invention environment-friendly and result in reduced pollution compared to prior art cooling plants and cleaning thereof.

According to pending regulations cooling plants for food should be cleaned daily and/or after each product shift. Any improvements of the cleaning of such cooling plant, in particular improvements relating to the reduction in treatment time, reduction in pollution and/or reduction in cost, are therefore a major benefit. In particular the saving in time for cleaning the equipment is beneficial since all non-production time is very costly.

In one embodiment the cooling chamber is an essentially closed chamber comprising a plurality of inner surfaces and one or more access openings. In practice it should preferably be possible to completely close the cooling chamber, since the formed foam will likely exit the cooling chamber through any available opening which may be undesired.

As indicated above the cooling plant may in principle have any size. Generally the blowing system of a cooling plant is adapted to the cooling plant and the size of the cooling chamber to obtain the desired rapid cooling, and accordingly it has been found that when applying the cleaning method of the invention merely the amount of treatment liquid used needs to be adapted to the size of the cooling chamber as explained further below.

Generally the cooling chamber should have an inner volume of at least about 1 m³ in order for the method to be truly cost effective. Whereas the method will also work for such small cooling plant, it may be simpler or as simple to clean such small cooling chambers manually. In one embodiment it is desired that the cooling chamber is 100 m³, such as at least about 1000 m³ or even larger. It has been found that in practice the larger the cooling chamber the more cost effective the cleaning method will be, at least up to a size of about 4000 m³.

The cooling plant of the invention may advantageously be an industrial cooling plant with a cooling chamber with a size of from about 100 to about 5000 m³. In principle there is no upper limit for the size of the cooling chamber, but in case of very large cooling chamber it may be preferred to clean the cooling chamber in two or more sections by applying a temporarily separating wall in the cooling chamber during the cleaning and/or additional blowers may be applied.

As mentioned above the one or more blowers should preferably be the entire or a part of a blowing system. The blower or blowers of the cooling plant may be any type of blower, such as any type of blower generally known to be used in cooling systems. The one or more blowers may for example be one or more of a positive displacement blower, a screw blower and a fan blower. The cooling plant preferably comprises a plurality of at least one of a positive displacement blower, a screw blower and a fan blower.

The skilled person knows the selection of blowers available for cooling plants and in practice any of these blowers are applicable in the present invention.

In one embodiment the blower comprises at least one fan such as a centrifugal fan or an axial fan.

In case the cooling plant comprises a blower which is constituted by a fan alone, i.e. the fan is not incorporated in a blower house, the blast exit is determined as the exit area of the fan where air leaves the fan to be blasted into the cooling chamber. It should be understood that the fan itself is arranged in the cooling chamber, i.e. the term “blasted into the cooling chamber” merely indicates that the fan provides additional energy to the air which is blasted further into the cooling chamber.

The distance between the blast exit and the hole structure is determined as the average of the length the gas (air) must pass from the blast exit to the first side of the hole structure.

In one embodiment where the blower comprises a blower house comprising the fan, the house comprises a nozzle with a nozzle exit, and the nozzle exit constitutes the blast exit. In this situation the blast exit accordingly has an exit area determined as the exit area of the nozzle exit.

The term ‘blast nozzle’ means a fixed or variable orifice constituting a supply end of the blower constructed to supply a continuous or modulated blast.

The cooling plant further comprises or is connected to a cooling plant with a cooling unit for cooling air such as it is generally known in the prior art cooling plants. In one embodiment the cooling plant comprises a cooling unit for cooling air, the cooling unit preferably being at least one of a heat exchanger, an evaporator or a compressor.

In one embodiment the cooling unit is integrated with one or more blowers. In another embodiment the cooling unit is separated from or displaced with respect to the one or more blowers. The cooling plant may of course comprise several cooling units of equal or different type.

The blast exit has an exit area which may in principle have any size and which will be selected in accordance with the size and type of cooling plant and cooling chamber and the number and arrangement of blast exits.

In one embodiment the blast exit has an exit area of at least about 5 cm², such as between about 10 cm² and about 10 m², such as up to 1 m². Larger blast exits may also be possible but often it will be more desirable to increase the number of blast exits than to provide blast exits larger than about 10 m² or even larger than 1 m².

In its first position, the screen covers at least a part of at least one blast exit.

In one embodiment the screen is such that in its first position it covers the blast exit, so that at least about 5% by volume of the gas supplied from the blast exit passes directly from the blast exit and through the hole structure, preferably at least about 10% by volume, such as at least about 30% by volume, such as at least about 60% by volume, such as 90% by volume such as essentially all of the gas supplied from the blast exit passes directly from the blast exit and through the hole structure.

The term “the gas passes directly from the blast exit and through the hole structure” preferably provides that the gas passes from the blast exit and through the hole structure without intermediate interference with any solid material other than the screen. Preferably nothing solid is arranged between the blast nozzel and the hole structure to interfere with the supplied (blasted) gas.

In its first position the screen should preferably cover the blast exit so that a sufficient amount of the gas supplied from the blast exit passes directly from the blast exit and through the hole structure so that the major part, preferably at least about 60% by vol., such as at least about 70% by vol., such as at least about 80% by vol. is foamed by the gas.

The screen may be fixed in its first position by any means, provided that it is stably fixed when the blower system is turned on and that the screen can be moved between its first and its second position as described below without damaging the screen or other parts of the cooling plant.

The screen may for example be mounted in one or more screen holders fixed adjacent, above, under and/or beside the blast exit. The screen may e.g. be mounted in such a holder by a click lock or any other temporary mounting methods.

In one embodiment the screen in its first position is fixed directly to the blower to at least partly cover the blast exit.

In one embodiment the screen in its first position is fixed with a screen distance to the blast exit which is sufficiently short to provide a desired gas flow through the hole structure. The screen distance may preferably be up to about 100 cm, such as up to about 50 cm, such as up to about 10 cm.

The screen distance is measured as the shortest distance between the blast exit and the screen.

In one embodiment the screen in its first position is fixed such that it has a blast exit-hole structure distance of up to about 100 cm, such as up to about 50 cm, such as up to about 10 cm.

The blast exit-hole structure distance is the average distance which the supplied gas passing through the hole structure must pass from the blast exit to the first surface of the hole structure.

The gas flow and the gas velocity through the hole structure should preferably be selected in relation to the total size of the second side of the hole structure and amount and type of treatment liquid to be applied in order to optimize the utility of the applied treatment liquid, i.e. such that as much as possible of the applied treatment liquid is foamed in the cleaning process.

In one embodiment the gas supplied from the blast exit has a supply velocity as supplied from the blast exit, and a first side velocity of the gas as it reaches the first side of the hole structure, the screen in its first position is fixed such that the first side velocity of the gas is at least about 50% of the supply velocity, such as at least about 75% of the supply velocity, such as up to about 100% of the supply velocity.

The supply velocity of gas supplied from the blast exit is the velocity immediately as supplied from the blast exit, i.e. at the point where the gas exits from the blast exit.

As indicated above the screen preferably comprises a second position. In its second position the screen is at least partly removed so that it does not to cover the blast exit.

In its second position the screen should preferably be removed from the blast exit such that it does not result in any pressure loss of gas supplied from the blast exit. Generally it will be highly undesired to let the screen remain in its first position during cooling operation as this will deteriorate the cooling effect and/or add to the energy consumption for providing the desired cooling.

The screen may be movable from its first position to its second position in any way. In one embodiment the screen is movably from its first position to its second position by a rotational movement, by a hinged movement and/or by a displaceable movement with respect to the blast exit.

In a preferred embodiment the screen is completely removable, preferably such that the screen can be withdrawn completely from the cooling chamber. Thereby it will also be possible to exchange the screen for example if it is damaged or if a screen with another hole structure is about to be used as it will be described further below.

The screen may have any structure provided that it comprises the hole structure. In one embodiment the screen is constituted by the hole structure. In another embodiment the screen comprises the hole structure with a frame or holder for the hole structure.

In one embodiment the screen comprises a tube section, for example such that the tube section provides a frame or holder for the hole structure. Thereby a very stable and handy screen is provided.

The tube section may have a first and a second end, the screen preferably being arranged such that the first end of the tube section is turned towards the blast exit and optionally is fixed to the blower when the screen is in its first position and the hole structure preferably is mounted at the second end of the tube section. It is desired that the tube section does not extend too long from the second side of the hole structure such as to make a foam worm in the exit part of the tube section (the end of the tube extending from the second side of the hole structure). What is too long can be determined by a simple test. Preferably the exit part of the tube section should exceed about 10 cm in length, such as up to 5 cm in length.

The tube section optionally has a cross-sectional area which differs from its first end to its second end, thereby the first side velocity of the gas may be optimized e.g. to be above 100% of the supplying velocity from the blast exit.

The purpose of the hole structure is to provide a substrate for supporting the treatment liquid such that it can be foamed by the gas passing through the hole structure. The hole structure should therefore preferably have as many liquid supporting surfaces as possible while simultaneously resulting in a relatively low resistance to the air flow. The hole structure has a first and a second side and a plurality of holes extending from the first to the second side where the holes in principle may have any size or sizes. However, in order to optimize the amount of the treatment liquid that is foamed the hole size should not be too large, as a too large hole size may cause too much of the treatment liquid to flow down to the floor of the cooling chamber without being foamed.

In one embodiment the hole structure in its first position is arranged such that the first surface thereof has an angle of up to about 20, preferably up to about 10 degrees from vertical, preferably the first surface thereof is substantially vertical. Thereby the hole structure provides a very good support for the treatment liquid.

The holes and directions of the holes through the hole structure may have any form. In one embodiment the hole structure has a hole direction between its first side and its second side which is substantially perpendicular to the first surface. In one embodiment the hole directions and/or hole size of the hole structure vary, e.g. through the thickness of the hole structure for example such that a hole structure layer closer to the first surface can act as a reservoir for a hole structure layer closer to the second surface of the hole structure. In one embodiment the first surface of the hole structure is essentially plane, in one embodiment the hole structure is concave e.g. with an inward-curving first surface.

The hole structure has a thickness defined as the distance between its first and its second side and an extension between its first side and its second side perpendicular to its thickness.

The hole structure may in one embodiment be essentially homogeneous in hole size over its thickness and extension. Generally it is desired that the flow resistance provided by the hole structure is essentially homogeneous over its extension, thereby the hole structure may be used optimally. However, in situations where the shape of the hole structure is concave the flow resistance provided by the hole structure may be lower in an inward-curving section than in the section surrounding the inward-curving section, thereby providing an optimal amount of blasted gas to be passed through the hole structure.

In one embodiment the thickness of the hole structure is substantially identical over its extension. The thickness may preferably be between about 0.1 mm and about 20 cm, such as between about 1 mm and about 10 cm. The optimal thickness depends largely on the type of material that it is made from and further a thicker hole structure may support more treatment liquid than a thinner hole structure.

The hole structure may be of any material with a sufficient mechanical strength. Examples of useful materials comprise perforated solid polymer, open foamed polymer, metal, textile and combinations thereof.

In one embodiment the hole structure is of a layered structure comprising two or more layers having equal or different hole sizes such as a hole size up to about 100 mm², the hole size of at least one of the layers being between about 0.1 μm² and about 25 mm², preferably between about 1 μm² and about 1 mm², more preferably all of the layers of the layered structure have a hole size of at least about 0.1 μm² and at least one of the layers has a hole size of up to about 25 mm².

The hole size is the average cross sectional area of holes in the hole structure or in a layer of the hole structure. In one embodiment the hole structure is shaped such that the average maximum cross dimension of a sectional cut through the hole structure is about 5 mm or less, such as about 2 mm or less, such as about 0.5 mm or more.

In situations where the hole structure is a porous structure the hole size is equivalent to the pore size.

In one embodiment the hole structure is of a single layer structure for example with a hole size up to about 100 mm², preferably between about 0.1 μm² and about 25 mm², such as between about 1 μm² and about 1 mm².

In one embodiment the hole structure comprises one or more layers of a wire lattice, preferably with a lattice distance of up to about 5 mm, such as from about 1 μm to about 2 mm or a wire mesh, preferably with an average mesh size between about 0.01 and about 5 mm, such as between about 0.1 and about 1 mm. The wire lattice should preferably be arranged with its lattice structure essentially horizontally to provide an optimal support for the treatment liquid.

The surface treatment liquid supply may be or comprise a simple container placed externally or internally of the cooling plant and/or its cooling chamber.

In one embodiment the surface treatment liquid supply is a liquid cavity into which the screen can be rotated for supplying a surface treatment liquid at least partly on the first side of the hole structure.

In one embodiment the surface treatment liquid supply comprises a supply inlet arranged to supply surface treatment liquid at least partly on the first side of the screen by applying surface treatment liquid at an upper part of the screen and allowing it to cover at least a part of the first side of the hole structure by gravity, e.g. as a ‘water fall’.

In one embodiment the surface treatment liquid supply comprises a supply inlet arranged to supply surface treatment liquid at least partly on the first side of the screen by spraying surface treatment liquid onto at least a part of the first side of the hole structure. The surface treatment supply may in this embodiment preferably comprise a spray nozzle fluidically connected to the supply inlet arranged to spray surface treatment liquid at least partly on the first side of the screen.

As mentioned above the cooling plant may be any type of cooling plants including freezing plants, chilling plants and combinations thereof.

In a preferred embodiment the cooling plant is a freezing plant for freezing ice cream.

In one embodiment the cooling plant comprises an article conveyer, such as a conveyer belt or a tray conveyer for carrying the article to be cooled in the cooling plant. In this embodiment it is preferred that the screen is arranged at a distance from the article conveyer which is larger than the distance between the blast exit and the screen. Thereby the risk of entrapping foam between the article conveyer and the screen is reduced. In case foam is entrapped between the article conveyer and the screen it may be collapsed prior to full utilization thereof.

In one embodiment where the cooling plant comprises an article conveyer, such as a conveyer belt or a tray conveyer for carrying the article to be cooled in the cooling plant, the screen is preferably arranged such that gas supplied from the blast exit has a minimum traveling distance to the article conveyer which is at least twice the shortest traveling distance to the screen, preferably the screen being arranged such that gas supplied from the blast exit has a minimum traveling distance to the article conveyer which is at least about 2.5 times, such as at least about 3 times, such as at least about 3.5 times the shortest traveling distance to the screen. Also in this embodiment the risk of undesired entrapping of foam is reduced.

The minimum traveling distance of the gas is measured as the straight traveling distance ignoring any change of direction due to resistance.

In one embodiment where the cooling plant comprises an article conveyer, such as a conveyer belt or a tray conveyer for carrying the article to be cooled in the cooling plant, the screen is arranged in a shortest distance from the article conveyer which is at least about 10 cm, such as at least about 20 cm, such as at least about 30 cm. Also in this embodiment the risk of undesired entrapping of foam is reduced.

The shortest distance is measured as the shortest distance in a direction perpendicular to the second surface of the screen.

In one embodiment the cooling plant is a batch cooling plant or an in-line cooling plant, such as an in-line continuous cooling plant.

In one embodiment the cooling plant is a freezer such as a blast freezer, for example a spiral freezer or a tunnel freezer. Such freezers are generally known in the art without the screen and the surface treatment liquid supply of the present invention.

A blast freezer is a freezer comprising one or more blowers e.g. fans and is arranged to circulate cold air typically from less than 0 to minus 25° C. over the product to be frozen, which product may for example be arranged on trays, racks or conveyers (tunnel belt(s)). Often the product is carried on conveyor belts through a horizontal tunnel or vertically in an ascending spiral.

Tunnel belt speed varies with product size and form. Small rockfish fillets for example might pass through a blast freezer in 25 minutes, while a whole 20-pound salmon might take four to six hours. The products may be packed or unpacked. In situations where it is unpacked it is particularly important that the freezer is cleaned daily.

In one embodiment the cooling plant comprises a plurality of blowers each comprising at least one blast exit. The cooling plant preferably comprises a blowing system comprising one or more, preferably a plurality of blast exits.

The cooling plant of the invention may comprise a plurality of at least partly removable screens each comprising a hole structure having a first and a second side, and each arranged such that in its first position it totally or partly covers a blast exit so that at least a part of the gas supplied from the blast exit passes directly from the blast exit and through the hole structure from the first side thereof. The cooling plant may comprises one or more surface treatment liquid supplies arranged to supply a surface treatment liquid at least partly on the first side of the respective hole structures.

The cooling plant of the invention may for example also comprise two or more surface treatment liquid supplies each individually of each other arranged to supply surface treatment liquid at least partly on the first side of one or more of the hole structures.

The invention also relates to a cooling plant system. The cooling plant system comprises
a cooling plant with a cooling chamber,
at least one blower comprising a blast exit,
at least one screen comprising a hole structure having a first and a second side, and
a surface treatment liquid supply,
   wherein the blower, the screen and the surface treatment liquid supply can be arranged such that the blower is capable of circulating gas in the cooling chamber by supplying gas from the blast exit, the screen in a first position totally or partly covers the blast exit so that at least a part of the gas supplied from the blast exit passes directly from the blast exit and through the hole structure from the first side thereof, and the surface treatment liquid supply supplies a surface treatment liquid at least partly on the first side of the hole structure.

The cooling plant of the cooling plant system may preferably be as described above.

In one embodiment of the cooling plant system at least one of the blower, the screen and the surface treatment liquid supply is removable from the cooling plant.

It is preferred that the blower or blowers are stationary and is/are of the blower system used in the cooling of the cooling plant. However, in certain situations e.g. for upgrading an existing cooling plant, the blower for foaming the treatment liquid may be a removable blower.

In one embodiment where the at least one screen is arranged to be completely removed in its second position it is desired that the cooling plant system comprises at least 2 screens having at least one property different from each other, such as a different hole structure, different shape, different material(s) and/or different size. These two or more screens may be used independently of each other e.g. for application of different treatment liquids. The number of screens may e.g. be 3 or even more.

In one embodiment the cooling plant system further comprises at least one treatment liquid.

The terms “treatment liquid” and “surface treatment liquid” are used interchangeably and include in principle any medium in liquid form such a liquids with one or more components, solutions and dispersions.

The cooling plant system preferably comprises at least one foaming treatment liquid.

A foaming treatment liquid is herein used to describe a treatment liquid which forms a foam using the Ross-Miles method described below, which foam has a life time of at least about 1 minutes.

In one embodiment the treatment liquid preferably has a half life time of from about 1 to about 10 minutes and a total life time of from about 2 to about 20 minutes.

The life time and half life time of the foam may be measured by withdrawing a sample of foam from the cooling plant immediately after it was prepared in a clear glass cylinder (9.5″×24″). The initial height is measured and the half life time is determined as the time it takes the height to drop to the half of the initial height (only the foam height is measured not including the liquid formed in the bottom of the glass cylinder). The life time of the foam is determined as the time it takes all the foam to collapse.

As an alternative the foam used may be foam generated according to the Ross-Miles method described below.

The foam life time should not be too long since this may result in an undesired filling level of the cooling chamber and furthermore it may take an undesirably long time long to empty the cooling chamber after the cleaning step. On the other hand, the foam should preferably have at least some foam stability in order to be effectively used. Preferred foam life time is from about 3 to about 10 minutes.

In one embodiment the cooling plant system comprises a defrosting liquid. The defrosting liquid may preferably be foamable. Alternatively the defrosting fluid may be vaporized by being applied on the first side of the hole structure where the hole structure is selected to have narrow holes.

In one embodiment the defrosting liquid is an aqueous solution optionally comprising a foaming agent e.g. a surface active component such as a surface tensioning lowering agent. Examples of foaming agents are fatty acids, alcohols, detergents, proteins, saponins and sulfates, such as sodium laureth sulfate, sodium lauryl ether sulfate (SLES), sodium lauryl sulfate (SLS) and ammonium lauryl sulfate (ALS).

The defrosting agent may for example comprise glycol.

In one embodiment the treatment liquid is a foamable cleaning liquid, preferably selected from an aqueous solution comprising at least one cleaning active compound.

The cleaning active compound optionally operates as a foaming agent.

In one embodiment the treatment liquid is a sanitizing liquid, preferably selected from an aqueous solution comprising one or more quaternary ammonium compounds.

In a preferred embodiment the treatment liquid is a combined cleaning and sanitizing liquid. Preferably the combined cleaning and sanitizing liquid is an aqueous solution comprising at least one quaternary ammonium compound and at least one cleaning active compound, where the quaternary ammonium compound and the cleaning active compound may be constituted by one or a combination of compounds.

The amount of quaternary ammonium compound in the combined cleaning and sanitizing liquid should preferably be relatively high in order to provide a desired fast and effective cleaning and sanitizing.

In one embodiment the treatment liquid comprises from about 0.1 to about 20% by weight of quaternary ammonium compound. A treatment liquid with a quaternary ammonium compound of 5% or higher is seldom used since in most situations a lower concentration of quaternary ammonium compound is sufficient. Preferred range of quaternary ammonium compound in the treatment liquid is from about 0.2 to about 5% by weight of quaternary ammonium compound.

The quaternary ammonium compound may preferably be a quaternary ammonium compound with the formula I

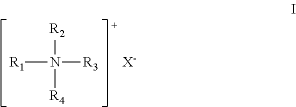

wherein R₁ and R₂ independently of one another represent alkyl radicals containing 1 to 4 carbon atoms or benzyl radicals, halogenated or alkylated benzyl radicals, or alkoxy groups, R₄ and R₅ independently of one another represent alkyl or benzyl radicals, halogenated or alkylated benzyl radicals containing 6 to 22 carbon atoms and X— is an anion, preferably from the groups of halides or carboxylates. Examples are dimethyl dioctyl ammonium chloride, didecyl dimethyl ammonium chloride, didodecyl dimethyl ammonium chloride, dimethyl ditetradecyl ammonium chloride, dihexadecyl dimethyl ammonium chloride, dimethyl dioctadecyl ammonium chloride, decyl dimethyl octyl ammonium chloride, dimethyl dodecyl octyl ammonium chloride, benzyl decyl dimethyl ammonium chloride, benzyl dimethyl dodecyl ammonium chloride, benzyl dimethyl tetradecyl ammonium chloride, decyl dimethyl (ethylbenzyl) ammonium chloride, decyl dimethyl(dimethyl benzyl)-ammonium chloride, (chlorobenzyl)-decyl dimethyl ammonium chloride, decyl-(dichlorobenzyl)-dimethyl ammonium chloride, benzyl didecyl methyl ammonium chloride, benzyl didocyl methyl ammonium chloride, benzyl ditetradecyl methyl ammonium chloride, benzyl dodecyl ethyl methyl ammonium chloride and the corresponding compounds which, instead of chloride, contain acetate or propionate as anions.

The quaternary ammonium compound may be prepared by any known method e.g. as described in U.S. Pat. No. 3,754,033 and U.S. Pat. No. 4,450,174.

In one embodiment the quaternary ammonium compound preferably is a dialkyl dimethyl quartenary ammonium compound.

The concentrations of quaternary ammonium compound are described by concentrations by weight. A weight % of 1% quaternary ammonium compound corresponds to about 10.000 parts per million (ppm).

The treatment liquid may preferably further comprise a solubility enhancing agent to ensure adequate solubility of the quaternary ammonium compound. The solubility enhancing agent may be present in up to about 20% by weight, such as from about 0.05 to about 10% by weight, such as from about 0.5 to about 5% by weight of the treatment liquid.

The solubility enhancing agent is any compatible solubility enhancing agent that solubilizes quaternary ammonium compound. The solubility enhancing agent is preferably selected from alcohols and/or polyglycols, such as polyethylene glycol. Alcohols are the preferred solubility enhancing agent. More preferably, the treatment liquid comprises one or more solubility enhancing agents selected from monohydric alcohols, dihydric alcohols, trihydric alcohols, and a combination thereof. Any one of these types of alcohols can be used alone or in combination with one or more of the other types of alcohols to obtain the desired solubility enhancing effect. If a monohydric alcohol is utilized, then this type of alcohol is preferably an aliphatic alcohol, and more preferably is ethyl alcohol. If a dihydric alcohol is utilized, then a glycol or a derivative thereof is preferred. Trihydric alcohols, such as glycerol or derivatives thereof, are also useful as a solubility enhancing agent in the present concentrated CPC solution. In a preferred embodiment the solubility enhancing agent is propan-2-ol.

The treatment liquid e.g. in the form of a combined cleaning and sanitizing liquid may preferably further comprise up to about 5% by weight of one or more components preferably selected from additives and surfactants. The remainder preferably is water.

The additives and surfactants may for example comprise one or more of chelators such as ethylenediaminetetraacetic acid (EDTA), sodium hydroxide for pH regulation, and dyes such as those commonly used in the art in cleaning and disinfecting solutions.

The cleaning and/or sanitizing liquid preferably has an initial foaming height of at least about 100 mm, preferably at least about 110 mm measured using Ross-Miles Foam Height Test (ASTM D1173-07) at 25° C.

Ross-Miles foam height measurement comprises using a sample of 200 ml of a test treatment liquid and drop it through a clear glass cylinder (9.5″×24″) impacted with 50 ml of the same treatment liquid. Due to the impacting force, foam will be generated and its height can be measured.

In one embodiment the treatment liquid is a water repellent liquid, preferably selected from polymers—e.g. in the form of a dispersion or a solution, wax, a wax dispersion, a surface tension reducing aqueous solution optionally comprising one or more of alkoholalkoxylate, sodium sulfonate and citrus acid.

In one embodiment the treatment liquid is selected such that upon application to the hole structure a major part of it can be foamed by gas supplied from the exit.

In one embodiment the treatment liquid is water rinse, such as tap water.

In one embodiment the treatment liquid is selected such that upon application to the hole structure it can be foamed by gas having a velocity of between about 0.5 m/sec and about 50 m/sec.

As indicated above, the invention also relates to a method of cleaning a cooling plant as described above. The method of the invention comprises
turning on the at least one blower;
applying the at least one screen in its first position to totally or partly cover at least one blast exit so that at least a part of the gas supplied from the blast exit passes directly from the blast exit and through the hole structure from the first side thereof;
applying a surface treatment liquid at least partly on the first side of the hole structure such that the treatment liquid is at least partly foamed;
allowing the at least one blower to operate in a treatment time of at least about 10 minutes, preferably at least about 15 minutes, such at up to about 2 hours, such as up to about 1 hours.

The treatment liquid need not be applied for the whole treatment time and often it will not be applied in the last minutes e.g. up to 15 minutes of the treatment time.

In one embodiment the treatment liquid is applied for at least about 25% of the treatment time, such as for at least 50% of the treatment time, such as for at least 75% of the treatment time.

The treatment liquid may be applied continuously or in steps, such as for example steps of at least about 30 seconds, such as at least about 2 minutes each.

The treatment liquid preferably should be applied such that a major amount of the treatment liquid will be foamed. Methods of applying the treatment liquid are described above. In a preferred embodiment the treatment liquid should preferably be applied with a velocity and spreading over the hole structure such that at least about 50% by volume, such as at least about 60%, such as at least about 70%, such as at least about 80% by volume of the treatment liquid is foamed. For optimizing the foaming effect the hole structure may preferably be selected to provide a good support for the treatment liquid as described above.

For optimizing the cleaning effect it is desired that substantially all of the blowers of the cooling plant are operating in at least 50% of the treatment time. Substantially all of the blowers mean herein preferably at least about 90% of the blowing capacity.

The treatment liquid may preferably be as described above and e.g. comprising one or more liquids selected from
a defrosting liquid;
a cleaning liquid;
a sanitizing liquid;
a combined cleaning and sanitizing liquid;
a water repellent liquid; and
water.

A defrosting liquid is particularly useful for treatment of freezing plants. In order to provide a good cleaning of the surfaces of the cooling chamber, the surfaces should preferably be free of ice. By applying a defrosting liquid as the treatment liquid, the inner surfaces of the cooling chamber of the freezing plant can be defrosted rapidly and thereby any ice can be rapidly removed from the surfaces. By using a foamable defrosting liquid the method of defrosting has shown to be surprisingly fast.

The defrosting liquid may for example be water optionally comprising a foaming additive for improving the contact time between the defrosting liquid and the inner surfaces of the cooling chamber in order to improve the energy transfer.

By applying a water repellent liquid after the cleaning and/or sanitizing and/or water treatment, the cooling plant can be rapidly emptied from liquid to a sufficient level whereby the cooling plant can be turned on to cooling operation without the risk of forming excessive amount of undesired iceing inside the cooling chamber. Water which is applied after the application of water repellent liquid will also be removed rapidly.

In one embodiment the method comprises treating the cooling plant with two or more treatment liquids, preferably in separate treatment steps. The screen may optionally be changed from one cleaning step to another.

In one embodiment the method comprises treating the cooling plant in separate steps with
a defrosting liquid;
a combined cleaning and sanitizing liquid;
a water repellent liquid; and
water.

### BRIEF DESCRIPTION OF DRAWINGS

The invention will be explained more fully below in connection with a preferred embodiment and with reference to the drawings in which:

FIG. 1a is a schematic cross-sectional cut through a batch freezer operating in a freezing mode.

FIG. 1b is the batch freezer of FIG. 1a operating in a cleaning mode.

FIG. 2a is a perspective view of a spiral freezer operating in a freezing mode and where a part of the wall and sealing of the cooling chamber have been removed.

FIG. 2b is the spiral freezer of FIG. 2a operating in a cleaning mode.

FIG. 3 is a perspective view of another spiral freezer where the walls of the cooling chamber are shown as transparent.

FIG. 4 is a perspective view of double spiral freezer where the walls of the cooling chamber are shown as transparent.

FIG. 5a is a perspective view of a two-level spiral freezer operating in a freezing mode and where a part of the wall and sealing of the cooling chamber have been removed.

FIG. 5b is the spiral freezer of FIG. 5a operating in a cleaning mode.

FIG. 6 is a perspective view of a freeze tunnel where a part of the wall and sealing of the cooling chamber have been removed and other parts are transparent.

FIG. 7 is a schematic cross-sectional cut through a chilling plant operating in a chilling mode.

FIG. 8 is a schematic cross-sectional cut through a cooling chamber operating in a cleaning mode.

FIG. 9a is a front view of the second side of a concave screen with a hole structure in the form of a network.

FIG. 9b is a cross-sectional view of the screen of FIG. 9a where the cut is taken in line A-A′.

FIG. 10a is a front view of the second side of a plane screen with a hole structure in the form of a porous material.

FIG. 10b is a cross-sectional view of the screen of FIG. 10a where the cut is taken in line B-B.

FIG. 11 is a front view of the second side of a plane screen with a hole structure in the form of a network and with a topside frame section for mounting in the cooling chamber.

FIG. 12 is a front view of the second side of a plane screen with a hole structure in the form of a perforated plate and with mounting elements for mounting in the cooling chamber.

FIG. 13 is a cross sectional view of a plane screen with a layered hole structure and a feeding spray head for feeding liquid to the screen.

The figures are schematic and may be simplified for clarity. Throughout the same reference numerals are used for identical or corresponding parts.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

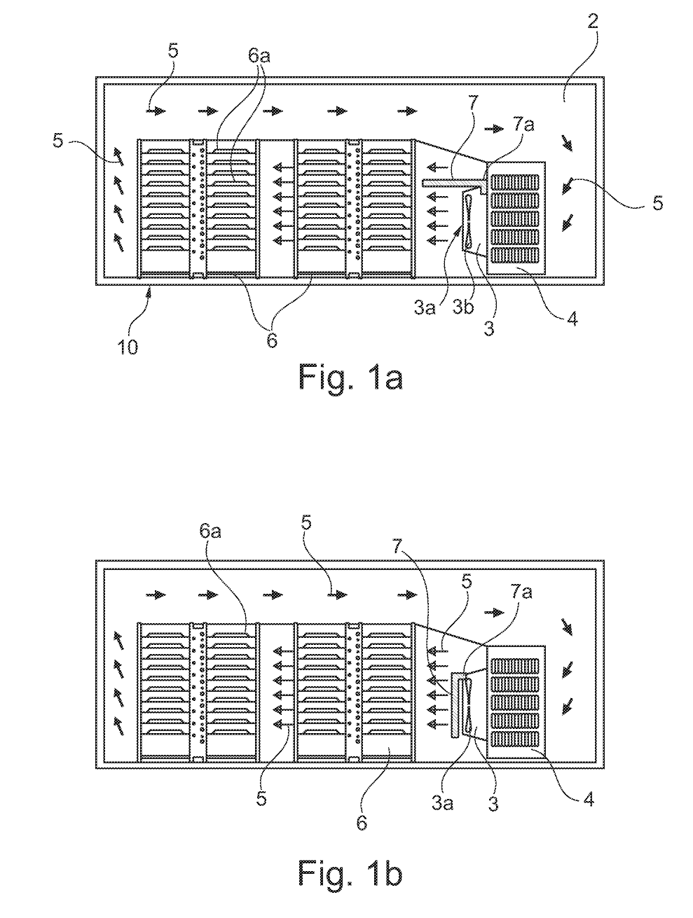

FIG. 1a is a schematic cross-sectional cut through a batch freezer operating in a freezing mode. The batch freezer 1 comprises a cooling chamber 2, which accordingly is a freezing chamber 2. In the freezing chamber 2, the batch freezer 1 comprises a cooling unit 4, a blower 3 with a fan blower 3b and a blast exit 3a. Furthermore a number of racks 6 carrying trays 6a for the product to be frozen are arranged in the batch freezer. The batch freezer further comprises a removed screen 7 comprising a hinge 7a and a not shown hole structure. The removed screen 7 is mounted to the cooling element 4 immediately above the blower 3 in a not shown mounting rail. Also a not shown liquid supply is arranged in the freezing chamber 2.

The arrows 5 show the flow directions and circulation of air in the freezing chamber 2. The air is cooled down in the cooling unit 4, and subsequently the blower 3 causes the air to pass through the racks 6 in order to cool and freeze the products carried on the trays 6a. Thereafter the air is circulated back to the cooling unit 4 for repeating the process. The removed screen 7 is arranged in its second position so that it does not cover the blast exit 3b and thereby does not interfere with the cooling process.

FIG. 1b shows the batch freezer 1 of FIG 1a operating in cleaning mode.

The cooling element 4 has been closed down so that the air is no longer being cooled and/or the cooling element 4 is set to reverse operation so that instead it heats the air.

The blower 3 is on, the screen 7 is moved along the not shown mounting rail and is bent in its hinge 7a to its first position so that it is immediately in front of the blast exit 3a with the first side of the hole structure facing the blast exit 3.

A cleaning liquid is fed to the first side of the hole structure and the cleaning is conducted as described above.

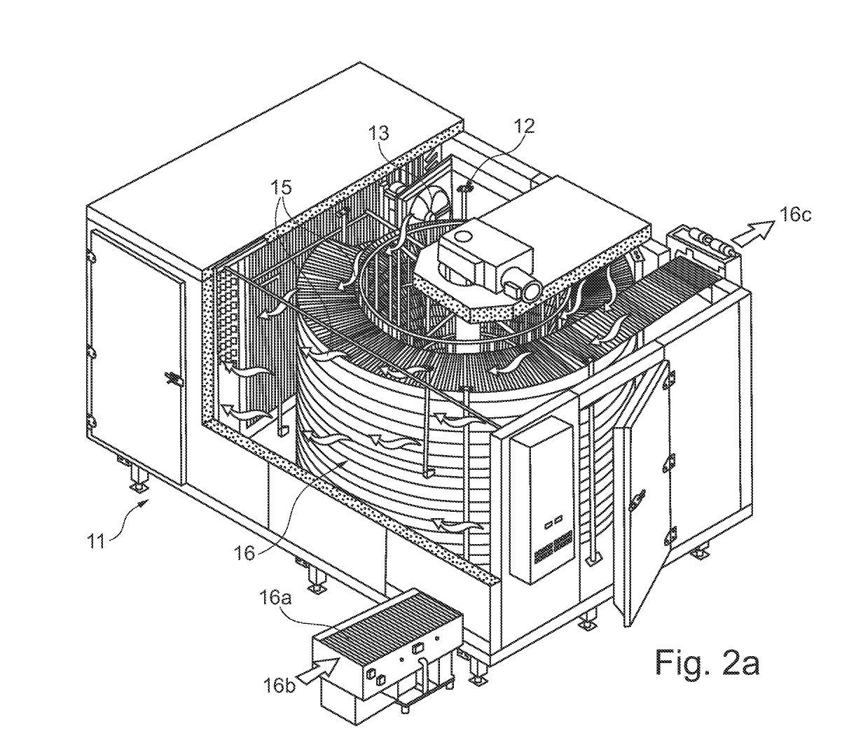

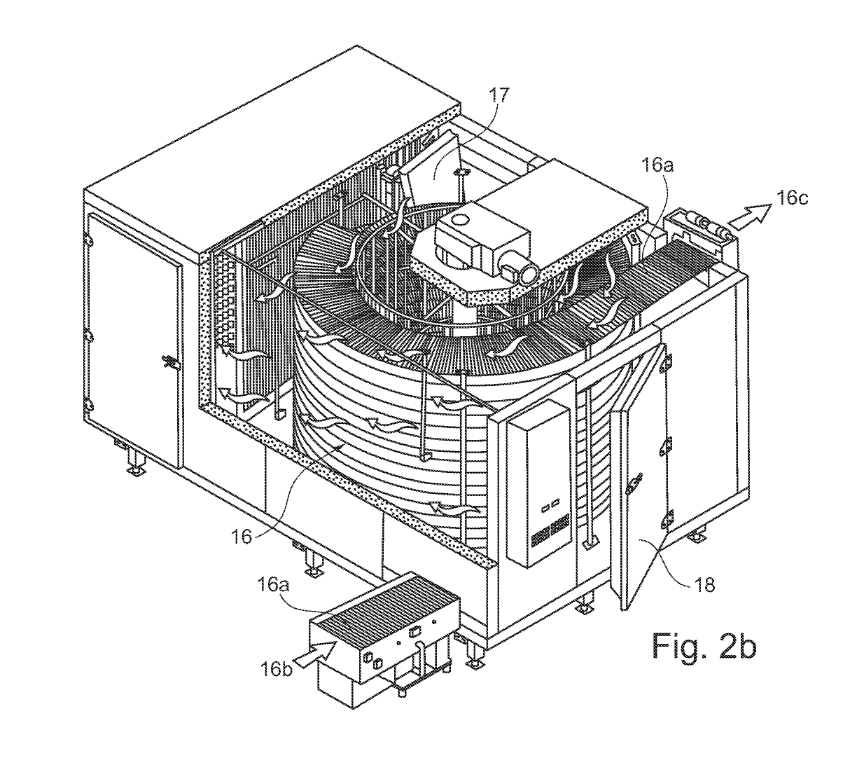

FIG. 2a shows a spiral freezer 11, with a freezing chamber 12. The spiral freezer 11 operates in a freezing mode. A part of the wall and sealing of the cooling chamber 12 have been cut out to see the inside thereof. The spiral freezer 11 comprises a cooling element 14 and a blower 13, as well as not shown mounting elements for a screen and a not shown liquid supply. The arrows 15 show the flow directions and circulation of air in the freezing chamber 12. The spiral freezer 11 is a continuous freezer type comprising a conveyer belt 16a, arranged in a spiral 16 inside the freezing chamber 12 and with an input site 16b for the food to be frozen and an exit site 16c for the frozen food. The food to be frozen is applied on the conveyer belt 16a at the input site 16b. The conveyer belt carries the food into the freezing chamber 12 into the spiral 16 where it is carried upwards in the spiralling circle for finally reaching the exit site 16c where the frozen food is withdrawn.

The freezing chamber comprises an access door 8 for inspection of the freezing production. The access door 8 should preferably remain closed during the cleaning process.

FIG. 2b shows the spiral freezer 11 of FIG. 2a operating in a cleaning mode.

The cooling element 14 has been closed down so that the air is no longer being cooled and/or the cooling element 14 is set to reverse operation so that instead it heats the air.

The conveyer belt 6a may or may not be stopped, but of course all the food should be withdrawn from the freezing chamber 12.

The blower 13 is on. A screen 17 carrying a not shown hole structure has been mounted immediately in front of the blower 13.

A cleaning liquid is fed to the side of the hole structure facing the blower 13 and the cleaning is conducted as described above.

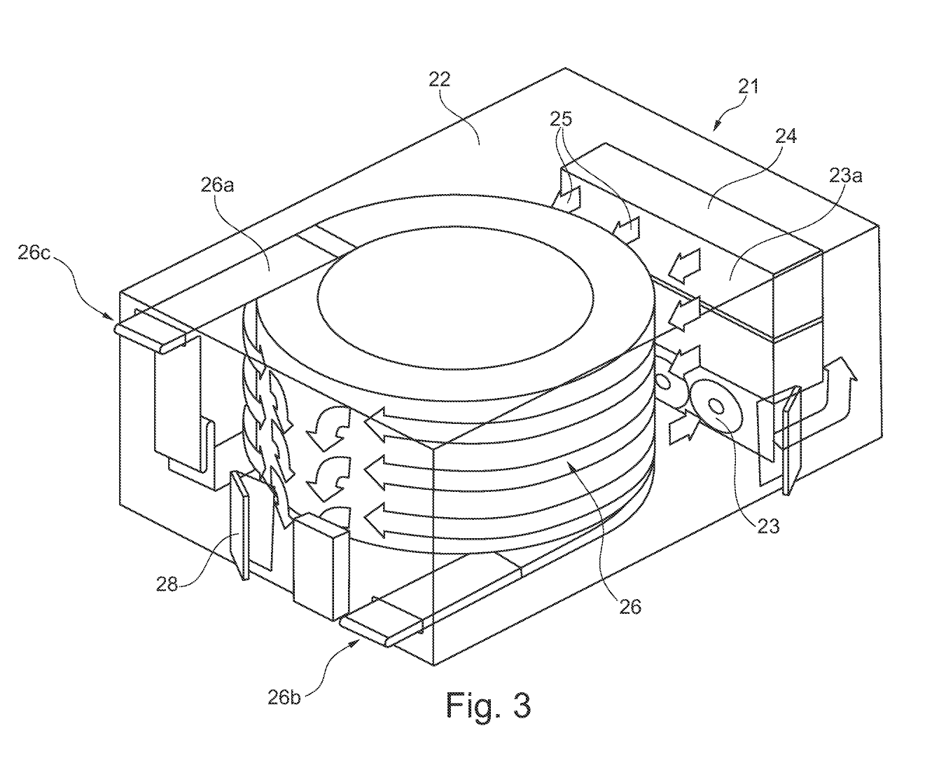

FIG. 3 is a schematic drawing of another spiral freezer 21. The spiral freezer 21 comprises a freezing chamber 22 with an access door 28 and a conveyer belt 26a, arranged in a spiral 26 inside the freezing chamber 22 and with an input site 26b for the food to be frozen and an exit site 26c for the frozen food. The spiral freezer 21 comprises a cooling element 24 and several blowers 23. The arrows 25 show the flow directions and circulation of air in the freezing chamber 22. The blowers 23 blow the air through the cooling element 24 and the air leaves the cooling element 24 through a blast exit 23a from where it is guided to the spiral 26 for freezing the food carried in the spiral. The spiral freezer 21 additionally comprises not shown mounting elements for a screen and a not shown liquid supply.

When operating in a cleaning mode a screen with a hole structure can be mounted in the not shown mounting elements so that the screen is arranged immediately in front of the blast exit 23a, and a cleaning liquid is fed to the side of the hole structure facing the blast exit 23a and the cleaning is conducted as described above.

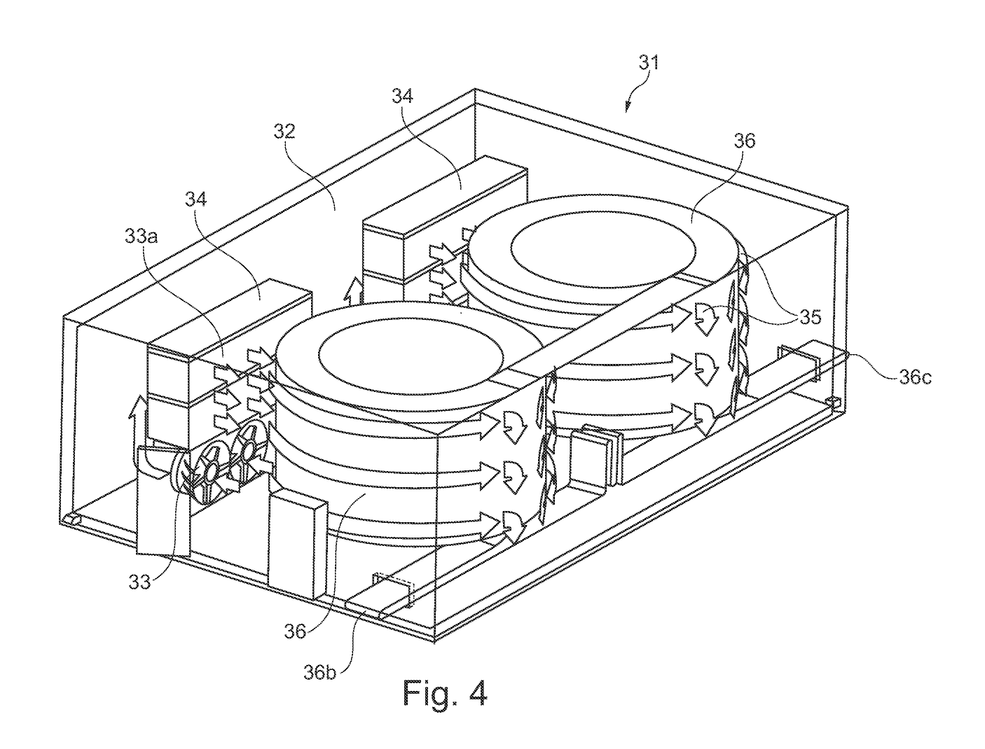

FIG. 4 is a schematic drawing of a double spiral freezer which is similar to the spiral freezer 21 shown in FIG. 3 with the exception that it comprises 2 spirals 36.

The spiral freezer 31 comprises a freezing chamber 32 with an access door 38 and a conveyer belt 36a, arranged in a double spiral 26 inside the freezing chamber 32 and with an input site 36b for the food to be frozen and an exit site 36c for the frozen food.

The food to be frozen is applied on the conveyer belt 36a at the input site 36b. The conveyer belt carries the food into the freezing chamber 32 into the first spiral 36 where it is carried upwards in the spiralling circle and further to the second spiral 36, where it is carried downwards for finally reaching the exit site 36c where the frozen food is withdrawn.

The spiral freezer 31 comprises two cooling elements 34 and several blowers 23. The arrows 35 show the flow directions and circulation of air in the freezing chamber 32. The blowers 23 blow the air through the cooling element 34 and the air leaves the cooling element 34 through a blast exit 23a from where it is guided to the spirals 26 for freezing the food carried in the spiral. The spiral freezer 31 additionally comprises not shown mounting elements for at least one screen and at least one not shown liquid supply.

When operating in a cleaning mode one or more screens with hole structures are mounted in not shown mounting elements so that the screens are arranged immediately in front of one or both of the blast exits 23a, and cleaning liquid is fed to the side of the respective hole structures facing the blast exits 23a and the cleaning is conducted as described above.

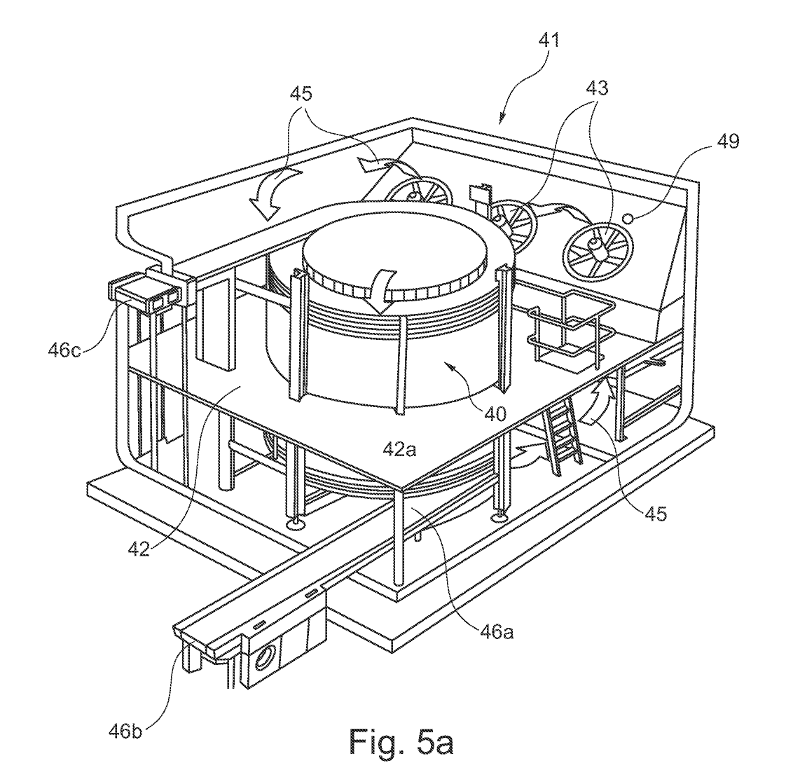

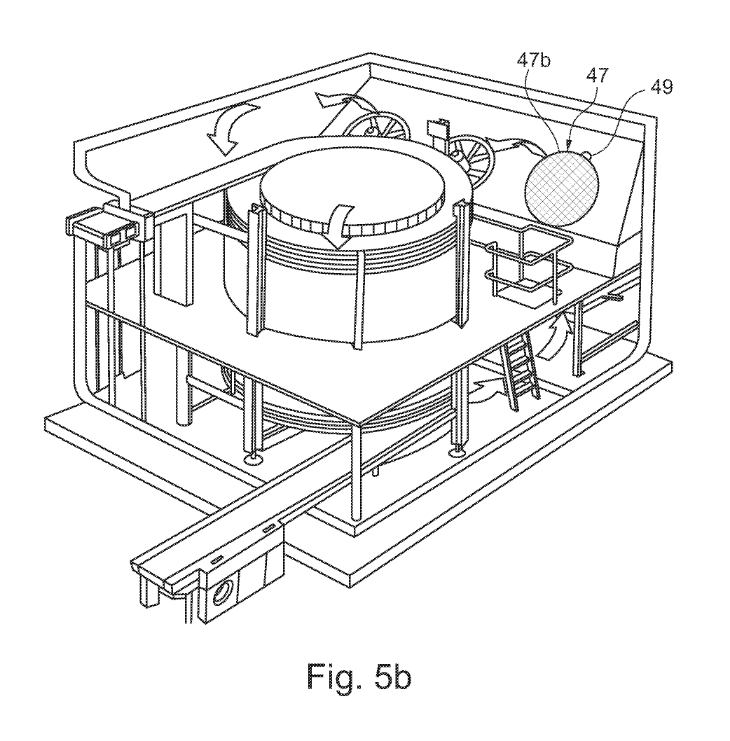

FIG. 5a is a perspective view of a two-level spiral freezer 41 operating in a freezing mode. The two-level spiral freezer 41 comprises a freezing chamber 42 in two levels, an upper and a lower level separated by a separation floor 42a.

The spiral freezer 41 comprises a conveyer belt 46a, arranged in a spiral 46 inside the freezing chamber 42 and with an input site 46b for the food to be frozen and an exit site 46c for the frozen food. The food to be frozen is applied on the conveyer belt 46a at the input site 46b. The conveyer belt carries the food into the freezing chamber 42 into the lower level and the spiral 46 where it is carried upwards to the upper level in the spiralling circle for finally reaching the exit site 46c where the frozen food is withdrawn.

The spiral freezer 41 comprises a not shown cooling element arranged below a number of blowers 43. The arrows 45 show the flow directions and circulation of air in the freezing chamber 42. Adjacent to one of the blowers 43 is not shown mounting elements for a screen and a liquid supply 49.

FIG. 5b shows the spiral freezer of FIG. 5a operating in a cleaning mode.

The cooling element is shut down. The conveyer belt 46a may or may not be stopped. The blowers 43 are on. A screen 47 carrying a hole structure 47b has been mounted immediately in front of one of the blowers 43.

A cleaning liquid is fed to the side of the hole structure facing the blower 43 via the liquid supply 49 and the cleaning is conducted as described above.

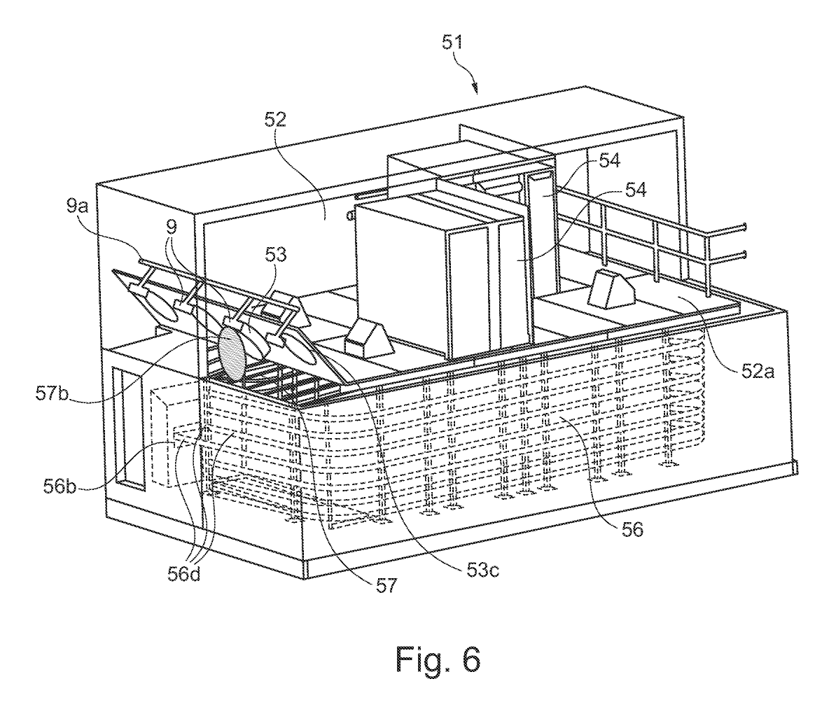

FIG. 6 is a perspective view of a continuous freeze tunnel 51. The freeze tunnel 51 comprises a freezing chamber 52 in two levels, an upper and a lower level separated by a separation floor 52a.

In its lower level, the freeze tunnel 51 comprises a pair of conveying bands 56 b for conveying not shown trays and/or a conveying band into a conveying structure 56 via an input site 56b. While passing through the conveying structure 56, food carried on the trays will be frozen and thereafter carried to a not shown exit site.

In its upper level, the freeze tunnel 51 comprises a pair of cooling elements 54 and a rack 53c arranged to hold up to 4 blowers 53. In the shown embodiment only one blower 53 is mounted. A screen 57 carrying a hole structure 57b is mounted in its second position above the blower 53. A liquid supply 59 is arranged above the screen 57. The liquid supply is a part of a liquid supply system comprising liquid supplies 59 for feeding liquid to up to 4 screens mounted in the rack 53c. The liquid supply system may be arranged to supply the same liquid to all of the liquid supplies 59 or in an alternative embodiment the liquid supply system may be arranged to supply different liquid from different liquid supplies 59.

For operating in a cleaning mode the screen 57 is moved to its first position so that it is arranged immediately in front of the blower 53. It may for example be held to the lowermost part of the blower 53 by a click lock. The blower is turned on and liquid is supplied from the liquid supply 59, which liquid supply disperses liquid like a waterfall on the first side of the hole structure facing the blower 53.

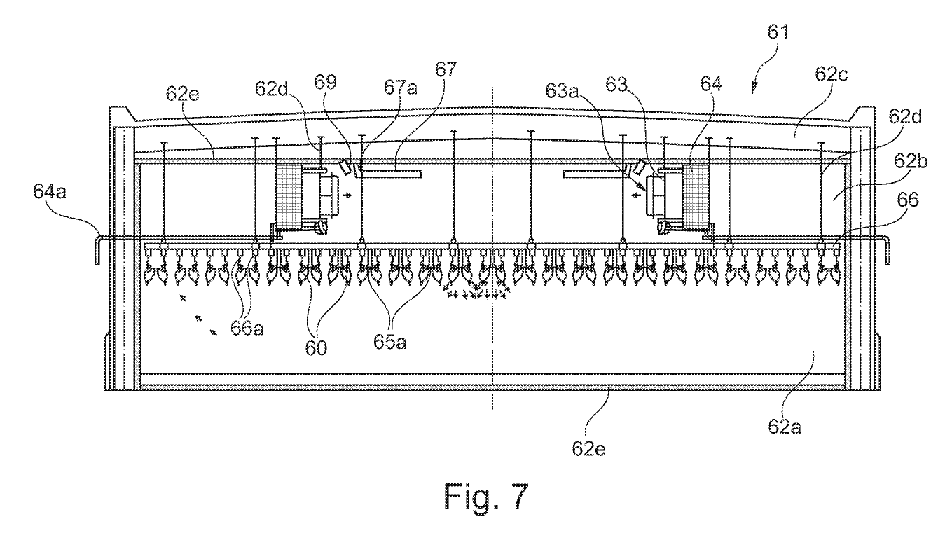

FIG. 7 is a schematic cross-sectional cut through a chilling plant 61 operating in a chilling mode. The chilling plant 61 comprises a chilling chamber 62a, 62b with an upper and a lower part partly separated by a conveyer structure 66 mounted to the ceiling 62c with mounting wires 62d or similar strong elements. In the upper level of the chilling chamber 62b is arranged a pair of air controlling elements each comprising a cooling element 64, a blower 63 and a blast exit 63a. Further a drain pipe 64a is arranged to guide away condensed water. The air controlling elements are mounted to the ceiling 62c by wires 62d or similar strong elements. A thermal insulation 62e is arranged to surround the air controlling elements and the lower level of the chilling chamber 62a.

The conveyer structure 66 comprises a conveying part and a stationary part. The conveying part of the conveyer structure carries a plurality of hooks 66a each holding a chicken to be chilled. The stationary part of the conveying structure 66 comprises a plurality of air guiding pipes 65a for guiding cold air from the upper level of the chilling chamber 62b towards the chickens to be chilled in the lower part of the chilling chamber 62a.

In the upper part of its chilling chamber 62b the chilling plant 61 further comprises two removed screens 67, each mounted to the insulation 62e immediately in front of the blast exit 63a of the respective air controlling elements. The screens 67 comprise each a hinge 67a and a not shown hole structure. The screens 67 can be moved to their first positions by being turned down such that they are arranged immediately in front of the respective blast exits 63a. A liquid supply 69 is arranged beside each if the screens 67 to apply a liquid to the first side of the screen 66 facing the blast exit 63a.

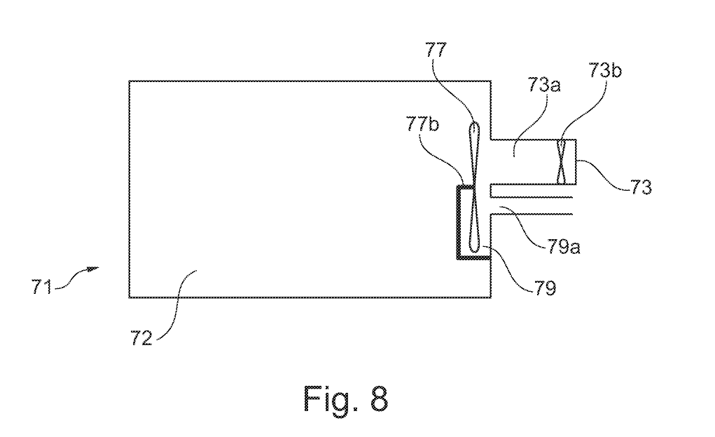

FIG. 8 is a schematic drawing of a cooling plant 71 with a cooling chamber 72 operating in a cleaning mode. The cooling plant 71 comprises a blower 73 with a fan 73b and a blast exit 73 leading blown air to the cooling chamber 72. Immediately in front of the blast exit 73a a screen 77 with a not shown hole structure is mounted. The screen 77 is mounted in a mounting 77b to an edge of a reservoir 79 providing a liquid supply 79 to the hole structure of the screen. Additional liquid can be fed to the liquid supply 79 via supply pipe 79a. The screen is rotated so that the lower half of the hole structure is dipped into the liquid supply 79 and the upper part of the hole structure is in front of the blast exit.

The screen may be manually removed after the cleaning has been terminated. Not used cleaning liquid may be drained off and a not shown lid may be applied to the liquid supply for avoiding contamination.

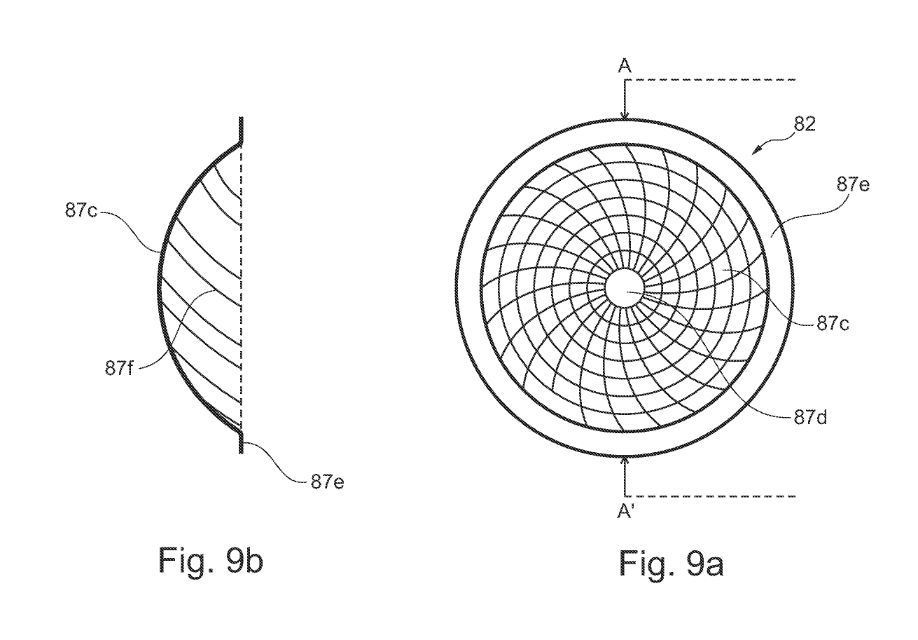

FIG. 9a is a front view of the second side 87c of a concave screen 87 with a hole structure in the form of a network. FIG. 9b is a cross-sectional view of the screen of FIG. 9a where the cut is taken in line A-A′.

The screen 87 comprises a frame 87e surrounding and holding the hole structure. The hole structure has a first side 87f and a second side 87c, both with a concave structure such that the thickness of the hole structure is substantially uniform over its extension. The hole sizes vary such that the holes are larger closer to the frame 87e and smaller closer to the top part 87d of the concave second surface 87c. Right at the top part 87d of the second surface 87c the hole structure may be essentially free of holes.

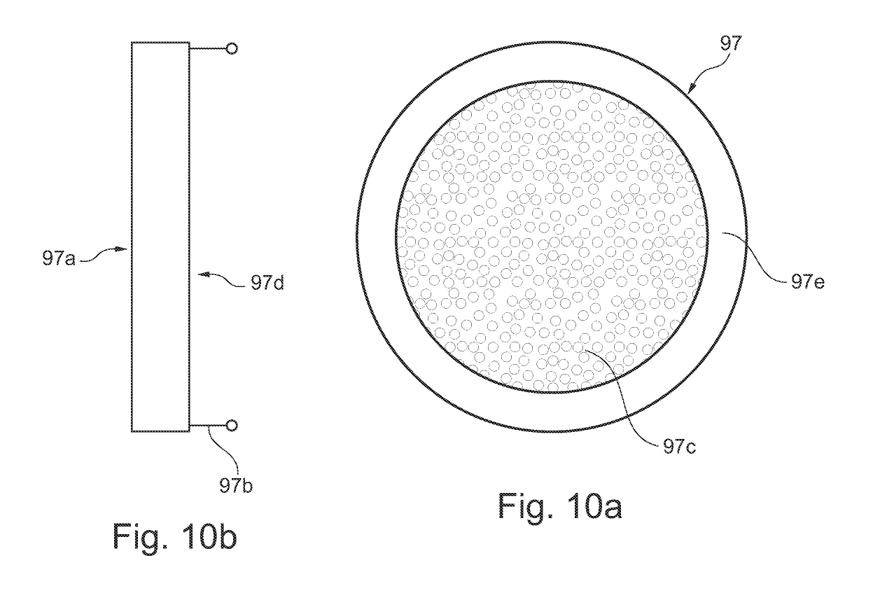

FIG. 10a is a front view of the second side 97c of a plane screen 97 with a hole structure in the form of a porous material. FIG. 10b is a cross-sectional view of the screen of FIG. 10a where the cut is taken in line B-B.

The screen comprises a frame 97e surrounding and holding the hole structure with the second surface 97c. The screen 97 comprises mounting elements 97b fixed to the frame 97e. The hole structure comprises a first surface 97f and a second surface 97c.

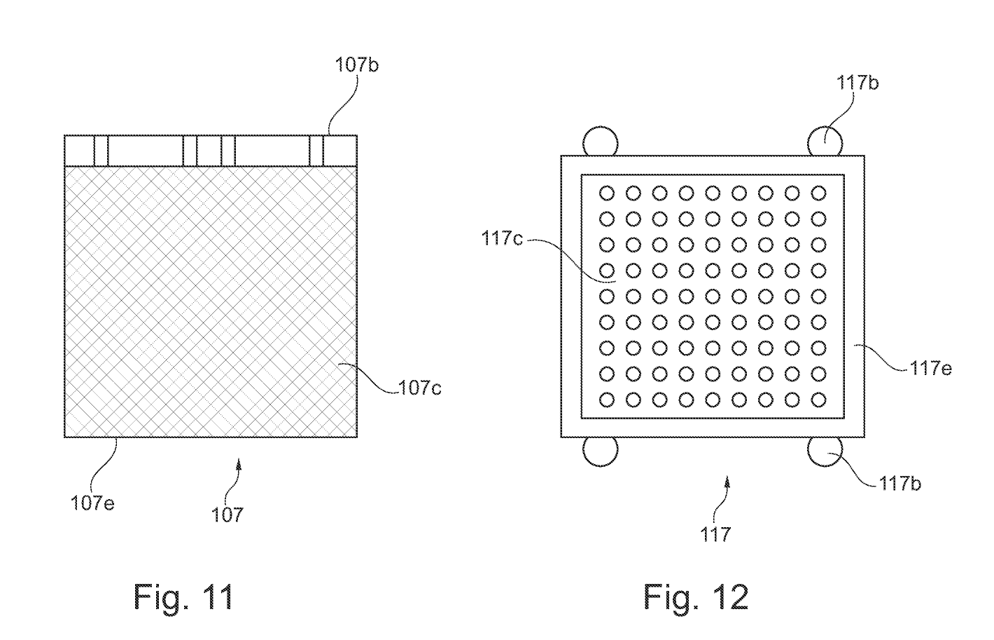

FIG. 11 is a front view of the second side 107c of a plane screen 107 with a hole structure in the form of a network held in a frame 107e and with a topside frame section 107b for mounting in the cooling chamber.

FIG. 12 is a front view of the second side 117c of a plane screen 117 with a hole structure in the form of a perforated plate held in a frame 117e and with mounting elements 117b for mounting in the cooling chamber.

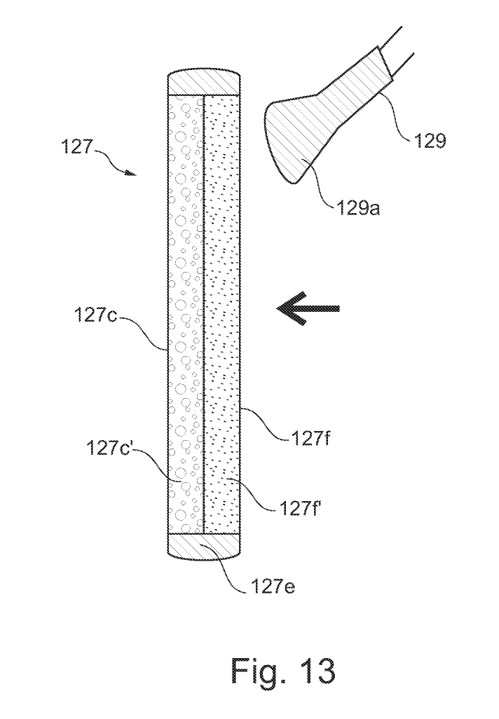

FIG. 13 is a cross sectional view of a plane screen 127 with a layered hole structure 127c′, 127f′. The hole structure 127c′, 127f′ is held in a frame 127e and comprises a first layer 127f′ providing a first surface 127f and a second layer 127c′ providing a second surface 127c. The first and the second surface have different hole structure. The first layer 127f′ provides a suitable reservoir for the second layer 127c′, and the second layer 127c provides a substrate for generating the foam. The drawing also shows a liquid supply 129 with a feeding spray head 129a for feeding liquid to the screen.

### EXAMPLE 1

Daily Cleaning of a Spiral Freezer in a Swedish Meat Production

A spiral freezer was used for freezing meat. In the cleaning process the cleaning chamber is emptied from meat and the cooling aggregates are shut off. The inner walls of the cooling chamber are defrosted using hot air and water.

After defrosting a removable screen and a cleaning liquid supply were mounted in front of one of the two permanent blowers.

The freezer band and the freezer blowers were started on full capacity.

The first side of the hole/perforated structure of the screen was then supplied with a 0.5% solution of a combined cleaning and sanitizing liquid, resulting in a foam production and foam release from the screen which was blasted into the cooling chamber covering all surfaces.

After approximately 10 minutes and 200 litres of cleaning solution, the first side of the hole structure of the screen is supplied with pure tap water, resulting in a water rinse of all the surfaces in the entire cooling chamber due to the water drops generated and released from the screen structure.

The water rinse takes approximately 20 minutes and uses approx. 400 litres of tap water, resulting in a total cleaning time of approx. 30 minutes.

Compared to previous manual cleaning of the same spiral freezer, the cleaning time was reduced to 5% (from 8 hours to 30 minutes), whereas the resource consumption (water, electricity and chemicals) was reduced to 10% compared to when using the known CIP cleaning method.

Furthermore, the hygienic result measured using ATP counts and bacterial number shows zero contamination on all the inner surfaces of the freezing plant.

### EXAMPLE 2

### Referring to FIG. 6

Weekly Cleaning of a Freeze Tunnel in a German Ice Cream Production

The freeze tunnel plant as shown in FIG. 6 was used for freezing ice cream.

Immediately after the last ice cream product had left the freezer, the removable screen and cleaning liquid supply were mounted in front of one of the four permanent blowers by a hinged movement.

The first side of the hole/perforated structure of the screen was then supplied with a 0.5% solution of a combined cleaning and sanitizing liquid, resulting in a foam production and foam release from the screen which was blasted into the cooling plant covering all surfaces.

After approximately 10 minutes and 200 litres of cleaning solution, the first side of the hole/perforated structure of the screen is supplied with pure tap water, resulting in a water rinse of all the surfaces in the entire cooling plant due to the water drops generated and released from the screen structure.

The water rinse takes approximately 20 minutes and uses approx. 400 litres of tap water, resulting in a total cleaning time of approx. 30 minutes. Compared to previous manual cleaning of the same tunnel freezer, the cleaning time has been reduced to below 20% (from 3 hours to 30 minutes), whereas the resource consumption (water, electricity and chemicals) has reduced to 25% compared to when using the known CIP cleaning method.

The hygienic result measured using ATP counts and bacterial number shows zero contamination on all the inner surfaces of the freezing plant.

Furthermore, the cleaning procedure also removes the dreaded “black spots” from the inner surfaces normally not removed using traditional cleaning.

### EXAMPLE 3

### Referring to FIG. 7

Daily Cleaning of a Cooling Tunnel in a Danish Poultry Production Plant

The chilling plant as shown in FIG. 7 was used for quick chilling of poultry.

After production a movable blower mounted with a screen and cleaning liquid supply is installed in the middle of the floor at the one end of the tunnel.

With all the permanent cooling blowers in full function, the first side of the screen of the movable blower is supplied with a 0.5% solution of a combined cleaning and sanitizing liquid, resulting in a foam production and foam release from the screen which was blasted into the lower compartment of the cooling tunnel. The action of all the permanent blowers together with the slow movement of the movable blower through the tunnel, results in a full coverage of all surfaces with foam.

After reaching the far end of the tunnel, the movable blower slowly returns to the start position during which the first side of the hole/perforated structure of the screen is supplied with pure tap water, resulting in a water rinse of all the surfaces in the entire cooling plant due to the water drops generated and released from the screen structure.

Each of the two steps in the cleaning cycle—foaming and water rinse—takes approximately 20 minutes, resulting in a total cleaning time of approx. 40 minutes, as compared to the normal 3-4 hours.

The hygienic result measured using ATP counts and bacterial number shows zero contamination on all the inner surfaces of the cooling plant.

The invention is defined by the features of the independent claim(s). Preferred embodiments are defined in the dependent claims. Any reference numerals in the claims are intended to be non-limiting for their scope.

Some preferred embodiments have been shown in the foregoing, but it should be stressed that the invention is not limited to these, but may be embodied in other ways within the subject-matter defined in the following claims.

1. A cooling plant comprising a cooling chamber and at least one blower comprising a blast exit for circulating gas in the cooling chamber, said cooling plant further comprising an at least partly removable screen and a surface treatment liquid supply, said removable screen comprising a hole structure having a first and a second side and a plurality of holes extending from the first to the second side, said screen being arranged such that in a first position it totally or partly covers the blast exit so that at least a part of the gas supplied from the blast exit passes directly from the blast exit and through the hole structure from the first side thereof, and said surface treatment liquid supply being arranged to supply a surface treatment liquid at least partly on the first side of said hole structure.

2. A cooling plant as claimed in claim 1 wherein the cooling chamber is an essentially closed chamber comprising a plurality of inner surfaces and one or more access openings.

3. A cooling plant as claimed in claim 1 wherein the cooling chamber has an inner volume of at least about 1 m³, such as at least about 100 m³, such as at least about 1000 m³.

4. A cooling plant as claimed in claim 1 wherein the blower is at least one of a positive displacement blower, a screw blower and a fan blower, the cooling plant preferably comprising a plurality of at least one of a positive displacement blower, a screw blower and a fan blower.

5. A cooling plant as claimed in claim 1 wherein the blower comprises at least one fan such as a centrifugal fan or an axial fan.

6. A cooling plant as claimed in claim 5, wherein the blower is constituted by the fan, the blast exit is determined as the exit area of the fan where gas, such as air leaves the fan to be blasted into the cooling chamber.

7. A cooling plant as claimed in claim 5, wherein the blower comprises a blower house comprising the fan, the house comprises a nozzle with a nozzle exit, said nozzle exit constitutes the blast exit, the blast exit has an exit area determined as the exit area of the nozzle exit.

8. A cooling plant as claimed in claim 1 wherein the cooling plant comprises a cooling unit for cooling gas, such as air, the cooling unit preferably being at least one of a heat exchanger, an evaporator, a compressor.

9. A cooling plant as claimed in claim 1 wherein the blast exit has an exit area of at least about 5 cm², such as between about 10 cm² and about 1 m².

10. A cooling plant as claimed in claim 1 wherein the screen in its first position covers the blast exit so that at least about 5% by volume of the gas supplied from the blast exit passes directly from the blast exit and through the hole structure, preferably at least about 10% by volume, such as at least about 30% by volume, such as at least about 60% by volume, such as 90% by volume such as essentially all of the gas supplied from the blast exit passes directly from the blast exit and through the hole structure.

11. A cooling plant as claimed in claim 1 wherein the screen in its first position is fixed directly to the blower to at least partly cover the blast exit.

12. A cooling plant as claimed in claim 1 wherein the screen in its first position is fixed with a screen distance to the blast exit, the screen distance being up to about 100 cm, such as up to about 50 cm, such as up to about 10 cm.

13. A cooling plant as claimed in claim 1 wherein the screen in its first position is fixed such that it has a blast exit-hole structure distance of up to about 100 cm, such as up to about 50 cm, such as up to about 10 cm.

14. A cooling plant as claimed in claim 1 wherein the gas supplied from the blast exit has a supplying velocity as supplied from the blast exit, and a first side velocity of the gas as it reaches the first side of the hole structure, the screen in its first position is fixed such that the first side velocity of the gas is at least about 50% of the supplying velocity, such as at least about 75% of the supplying velocity, such as up to about 100% of the supplying velocity.

15. A cooling plant as claimed in claim 1 wherein the screen comprises a second position where it is at least partly removed so that it does not to cover the blast exit, the screen preferably being movable from its first position to its second position by a rotational movement, by a hinged movement and/or by a displaceable movement with respect to the blast exit.

16. A cooling plant as claimed in claim 1 wherein the screen is completely removable, preferably the screen can be withdrawn from the cooling chamber.

17. A cooling plant as claimed in claim 1 wherein the screen is constituted by the hole structure or by the hole structure with a frame or holder for the hole structure.

18. A cooling plant as claimed in claim 1 wherein the screen comprises a tube section, the tube section preferably providing a frame or holder for the hole structure.

19. A cooling plant as claimed in claim 1 wherein the hole structure has a thickness defined as the distance between its first and its second side and an extension between its first side and its second side, the thickness of the hole structure varies over its extension preferably the first and/or the second side of the hole structure being concave or convex.

20. A cooling plant as claimed in claim 1 wherein the hole structure has a thickness defined as the distance between its first and its second side and an extension between its first side and its second side, the thickness of the hole structure is substantially identical over its extension, the thickness preferably being between about 0 1 mm and about 20 cm, such as between about 1 mm and about 10 cm.

21. A cooling plant as claimed in claim 1 wherein the hole structure is of material selected from perforated solid polymer, open foamed polymer, metal, textile and combinations thereof.

22. A cooling plant as claimed in claim 1 wherein the hole structure is of a layered structure comprising two or more layers having equal or different hole sizes such as a hole size up to about 100 mm², the hole size of at least one of the layers being between about 0.1 μm² and about 25 mm², preferably between about 1 μm² and about 1 mm², more preferably all of the layers of the layered structure have a hole size of at least about 0.1 μm² and at least one of the layers has a hole size of up to about 25 mm².

23. A cooling plant as claimed in claim 1 wherein the hole structure is of a single layer structure for example with a hole size of up to about 100 mm², preferably between about 0.1 μm² and about 25 mm², such as between about 1 μm² and about 1 mm².

24. A cooling plant as claimed in claim 1 wherein the hole structure comprises one or more layers of a wire lattice, preferably with a lattice distance of up to about 5 mm, such as from about 1 μm to about 2 mm or a wire mesh, preferably with an average mesh size between about 0.01 and about 5 mm, such as between about 0.1 and about 1 mm

25. A cooling plant as claimed in claim 1 wherein the surface treatment liquid supply is a liquid cavity into which the screen can be rotated for supplying a surface treatment liquid at least partly on the first side of said hole structure.

26. A cooling plant as claimed in claim 1 wherein the surface treatment liquid supply comprises a supply inlet arranged to supply surface treatment liquid at least partly on the first side of said screen by applying surface treatment liquid at an upper part of the screen and allowing it to cover at least a part of the first side of said hole structure by gravity.

27. A cooling plant as claimed in claim 1 wherein the surface treatment liquid supply comprises a supply inlet arranged to supply surface treatment liquid at least partly on the first side of said screen by spraying surface treatment liquid onto at least a part of the first side of said hole structure, the surface treatment supply preferably comprising a spray nozzle fluidically connected to said supply inlet arranged to spray surface treatment liquid at least partly on the first side of said screen.

28. A cooling plant as claimed in claim 1 wherein the cooling plant comprises an article conveyer, such as a conveyer belt or a tray conveyer for carrying the article to be cooled in the cooling plant, said screen being arranged at a distance from said article conveyer which is larger than the distance between said blast exit and said screen.

29. A cooling plant as claimed in claim 1 wherein the cooling plant comprises an article conveyer, such as a conveyer belt or a tray conveyer for carrying the article to be cooled in the cooling plant, said screen being arranged such that gas supplied from said blast exit has a minimum traveling distance to the article conveyer which is at least twice the shortest traveling distance to the screen, preferably the screen being arranged such that gas supplied from said blast exit has a minimum traveling distance to the article conveyer which is at least about 2.5 times, such as at least about 3 times, such as at least about 3.5 times the shortest traveling distance to the screen.

30. A cooling plant as claimed in claim 1 wherein the cooling plant comprises an article conveyer, such as a conveyer belt or a tray conveyer for carrying the article to be cooled in the cooling plant, said screen being arranged with a shortest distance from said article conveyer which is at least about 10 cm, such as at least about 20 cm, such as at least about 30 cm.

31. A cooling plant as claimed in claim 1 wherein the cooling plant is a batch cooling plant or an in-line cooling plant, preferably the cooling plant is an in-line continuous cooling plant.

32. A cooling plant as claimed in claim 1 wherein the cooling plant is a freezer, such as spiral freezer or a tunnel freezer.

33. A cooling plant as claimed in claim 1 wherein the cooling plant comprises a plurality of blowers each comprising at least one blast exit.

34. A cooling plant as claimed in claim 33 wherein the cooling plant comprises a plurality of at least partly removable screens each comprising a hole structure having a first and a second side, and each arranged such that in a first position it totally or partly covers a blast exit so that at least a part of the gas supplied from said blast exit passes directly from said blast exit and through said hole structure from the first side thereof, one or more surface treatment liquid supplies being arranged to supply a surface treatment liquid at least partly on the first side of said respective hole structures.

35. A cooling plant as claimed in claim 34 wherein the cooling plant comprises two or more surface treatment liquid supplies each individually of each other arranged to supply surface treatment liquid at least partly on the first side of one or more of said hole structures.

36. A cooling plant system comprising
a cooling plant with a cooling chamber,
at least one blower comprising a blast exit least,
at least one screen comprising a hole structure having a first and a second side, and
a surface treatment liquid supply,
the blower, the screen and the surface treatment liquid supply can be arranged such that the blower is capable of circulating gas in the cooling chamber by supplying gas from the blast exit, the screen in a first position totally or partly covers the blast exit so that at least a part of the gas supplied from the blast exit passes directly from the blast exit and through the hole structure from the first side thereof, and the surface treatment liquid supply supplies a surface treatment liquid at least partly on the first side of said hole structure.

37. A cooling plant system using the cooling plant of claim 1.

38. A cooling plant system as claimed in claim 36, wherein at least one of the blower, the screen and the surface treatment liquid supply is removable from the cooling plant.

39. A cooling plant system as claimed in claim 36, wherein the cooling plant system comprises a plurality of at least one of the blower, the screen and the surface treatment liquid supply.

40. A cooling plant system as claimed in claim 36, wherein the cooling plant system comprises at least 2 screens having at least one property different from each other, such as a different hole structure, different shape, different material(s) and/or different size.

41. A cooling plant system as claimed in claim 36, wherein the cooling plant system further comprises at least one treatment liquid.

42. A cooling plant system as claimed in claim 41 wherein the treatment liquid is a defrosting liquid, preferably the defrosting liquid being foamable and/or hot water, such as hot water comprising a foaming agent.

43. A cooling plant system as claimed in claim 41 wherein the treatment liquid is a cleaning liquid, preferably selected from an aqueous solution comprising at least one cleaning active compound, the cleaning liquid preferably being foamable.

44. A cooling plant system as claimed in claim 41 wherein the treatment liquid is a sanitizing liquid, preferably selected from an aqueous solution comprising one or more quaternary ammonium compounds.

45. A cooling plant system as claimed in claim 43 wherein the treatment liquid is a combined cleaning and sanitizing liquid, preferably the treatment liquid is a an aqueous solution comprising at least one quaternary ammonium compound and at least one cleaning active compound, where the quaternary ammonium compound and the cleaning active compound may be constituted by one or a combination of compounds.

46. A cooling plant system as claimed in claim 45 wherein the treatment liquid is a combined cleaning and sanitizing liquid, comprising from about 0.1 to about 20% by weight of quaternary ammonium compound; up to about 20% by weight of solubility enhancing agent; up to about 5% by weight of one or more components selected from additives and surfactants and water, the quaternary ammonium compound preferably being a dialkyl dimethyl quartenary ammonium compound.

47. A cooling plant system as claimed in claim 43 wherein said cleaning and/or sanitizing liquid has an initial foaming height of at least about 100 mm, preferably at least about 110 mm measured using Ross-Miles Foam Height Test (ASTM D1173-07) at 25° C.

48. A cooling plant system as claimed in claim 41 wherein the treatment liquid is a water repellent liquid, preferably selected from polymers, wax, a wax dispersion, a surface tension reducing aqueous solution optionally comprising one or more of alkoholalkoxylate, sodium sulfonate and citrus acid.

49. A cooling plant system as claimed in claim 41 wherein the treatment liquid is selected such that upon application to the hole structure it can be foamed by gas supplied from said exit.

50. A cooling plant system as claimed in claim 41 wherein the treatment liquid is water rinse, such as tap water.

51. A cooling plant system as claimed in claim 36 wherein the treatment liquid is selected such that upon application to the hole structure it can be foamed by gas having a velocity of between about 0.5 m/sec and about 50 m/sec.

52. A method of cleaning a cooling plant as claimed in claim 1, the method comprising
turning on the at least one blower;
applying the at least one screen in its first position to totally or partly cover at least one blast exit so that at least a part of the gas supplied from the blast exit passes directly from the blast exit and through the hole structure from the first side thereof;
applying a surface treatment liquid at least partly on the first side of said hole structure such that the treatment liquid is at least partly foamed;
allowing the at least one blower to operate in a treatment time of at least about 10 minutes, preferably at least about 15 minutes, such at up to about 2 hours, such as up to about 1 hours.

53. A method of cleaning a cooling plant as claimed in claim 52 wherein the treatment liquid is applied for at least about 50% of the treatment time, preferably the treatment liquid is applied continuously or in steps of at least about 30 seconds, such as at least about 2 minutes.

54. A method of cleaning a cooling plant as claimed in claim 52 wherein the treatment liquid is applied at a velocity and spreading over the hole structure such that at least about 50% by volume, such as at least about 60%, such as at least about 70%, such as at least about 80% by volume of the treatment liquid is foamed.

55. A method of cleaning a cooling plant as claimed in claim 52 wherein substantially all of the blowers of the cooling plant are operating in at least about 50% of the treatment time.

56. A method of cleaning a cooling plant as claimed in claim 52 wherein the treatment liquid is a cleaning liquid, preferably selected from an aqueous solution comprising at least one cleaning active compound, the cleaning liquid preferably being foamable.

57. A method of cleaning a cooling plant as claimed in claim 52 wherein the treatment liquid is a sanitizing liquid, preferably selected from an aqueous solution comprising one or more quaternary ammonium compounds.

58. A method of cleaning a cooling plant as claimed in claim 52 wherein the treatment liquid is a combined cleaning and sanitizing liquid, preferably the treatment liquid is a an aqueous solution comprising at least one quaternary ammonium compound and at least one cleaning active compound, where the quaternary ammonium compound and the cleaning active compound may be constituted by one or a combination of compounds.

59. A method of cleaning a cooling plant as claimed in claim 58 wherein the treatment liquid is a combined cleaning and sanitizing liquid, comprising from about 0.1 to about 20% by weight of quaternary ammonium compound; up to about 20% by weight of solubility enhancing agent; up to about 5% by weight of one or more components selected from additives and surfactants and water, the quaternary ammonium compound preferably being a dialkyl dimethyl quartenary ammonium compound.

60. A method of cleaning a cooling plant as claimed in claim 56 wherein said cleaning and/or sanitizing liquid has an initial foaming height of at least about 100 mm, preferably at least about 110 mm measured using Ross-Miles Foam Height Test (ASTM D1173-07) at 25° C.

61. A method of cleaning a cooling plant as claimed in claim 52 wherein the method comprises treating the cooling plant with two or more treatment liquids, preferably in separate treatment steps.

62. A method of cleaning a cooling plant as claimed in claim 61 wherein the method comprises changing the screen between treatments with different treatment liquids.

63. A method of cleaning a cooling plant as claimed in claim 52 wherein the method comprises treatment with one or more liquids selected from
a defrosting liquid;
a cleaning liquid;
a sanitizing liquid;
a combined cleaning and sanitizing liquid;
a water repellent liquid; and
water.
